(19) United States
(12) Patent Application Publication
(10) Pub. No.: US 2011/0091540 A1
(43) Pub. Date: Apr. 21, 2011

(54) POLYMERIC COMPOSITIONS AND THEIR METHOD OF USE IN COMBINATION WITH ACTIVE AGENTS

(76) Inventors: Alan Strickland, Lake Jackson, TX (US); George Grass, Tahoe City, CA (US)

(21) Appl. No.: 12/675,698

(22) PCT Filed: Aug. 29, 2008
(86) PCT No.: PCT/US2008/074848

§ 371 Date: Jan. 6, 2011

Related U.S. Application Data
(60) Provisional application No. 60/968,818, filed on Aug. 29, 2007.
(60) Provisional application No. 60/968,820, filed on Aug. 29, 2007.
(60) Provisional application No. 60/968,821, filed on Aug. 29, 2007.

(51) Int. Cl. A61K 31/78; A61K 9/48; A61K 9/52; A61P 9/00; A61P 13/12

(52) U.S. Cl. 424/457; 424/78.31; 424/463; 424/451

(57) ABSTRACT

A combination of active agents is disclosed which are particularly useful for treating fluid overload conditions. Methods for using the combination of active agent are also disclosed. The combination can include a highly absorbent polyelectrolyte polymer along with an agent that increases the amount of fluid in the intestine.

### FIELD OF THE DISCLOSURE

The present disclosure relates generally to combinations of active agents. The compositions can include highly absorbent polyelectrolyte polymers with high saline holding capacity and agents that increase the amount of fluid in the intestine. The agents can be mixed and administered in a single unitary dosage form or can be administered separately such that they will act in concert. Methods are also provided for removing fluid and/or modulating ion levels in a subject by administering a cross-linked polyelectrolyte polymer with an agent that increases the amount of fluid in the intestine.

### BACKGROUND

Numerous diseases and disorders are associated with increased retention of fluid (e.g., congestive heart failure and end stage renal disease (ESRD) and/or with ion imbalances (e.g., hyperkalemia, hypercalcemia, hyperphosphatemia and hyperoxalemia). For example, patients afflicted with retention of fluid often suffer from edema (e.g. pulmonary edema and/or edema of the legs) and the buildup of waste products in the blood (e.g., urea, creatinine, other nitrogenous waste products, and electrolytes or minerals, such as sodium, phosphate and potassium). Additionally, patients afflicted with an increased level of potassium may exhibit a variety of symptoms ranging from malaise, palpitations, muscle weakness and in severe cases, cardiac arrhythmias. Also, for example, patients afflicted with increased levels of sodium (e.g., hypernatremia) may exhibit a variety of symptoms including, lethargy, weakness, irritability, edema and in severe cases, seizures and coma.

Treatments for diseases or disorders associated with an increased retention of fluid (e.g., fluid overload) and/or ion imbalances attempt to decrease the retention of fluid and restore the ion balance. For example, treatment of diseases or disorders associated with ion imbalances may employ the use of ion exchange resins to restore ion balance. Treatment of diseases or disorders associated with an increased retention of fluid may involve the use of diuretics (e.g., administration of diuretic agents and/or dialysis, such as hemodialysis or peritoneal dialysis) and remediation of waste products that accumulate in the body. Additionally or alternatively, treatment for ion imbalances and/or increased retention of fluid may include restrictions on dietary consumption of electrolytes and water. However, the effectiveness and/or patient compliance with present treatments is less than desired and therefore often ineffective.

### SUMMARY

Combinations of active agents are disclosed which are particularly useful for treating fluid overload conditions. Methods for using the combination of active agents are also disclosed. The combination can include a highly absorbent polyelectrolyte polymer along with an agent that increases the amount of fluid in the intestine. The agents can be mixed and administered in a single unitary dosage form or can be administered separately such that they will act in concert in a subject.

In an exemplary method a subject is identified that has a fluid overload condition. A polyelectrolyte polymer that absorbs about 20-fold, 40-fold or more of its mass in physiological saline is obtained and administered to the intestine of a subject and an agent that increases the fluid present in the small intestine of the subject can also be administered.

The polyelectrolyte polymer can be a crosslinked polyelectrolyte such as polyacrylic acid or a crosslinked methacrylic acid having from about 0% to 95% of its acid groups bound with a counterion, including a cation such as sodium.

Any agent that brings about increased fluid in the intestine can be used in the present disclosure. Suitable agents include, for example, non-fermenting osmotic agents including polyethylene glycol having a molecular weight between 400 and 10,000 Daltons, non-absorbed sugar derivatives (e.g., those that are not significantly fermented under the conditions of use), poorly absorbed salts, inhibitors of intestinal sodium absorption, agents that increase sodium secretion into the intestine, agents that induce chloride secretion into the intestine, irritants or their mixtures.

Additional features and advantages are described herein, and will be apparent from, the following Detailed Description.

### DETAILED DESCRIPTION

Cross-linked polyelectrolyte polymers that absorb greater than 20 times their mass in saline (e.g., superabsorbent polymers) can be used in combination with one or more agents to treat diseases or disorders associated with fluid retention (e.g., fluid overload state) and/or ion imbalance. Cross-linked polyelectrolyte polymers that absorb greater than 20-fold, 30-fold, 40-fold, 50-fold, 60-fold, 70-fold, 80-fold, 90-fold, 100-fold or 110-fold or more of their mass in aqueous saline solution may be administered to a subject to remove fluid and/or modulate ion levels in the subject. Such polymers may vary in their counterion content.

The present disclosure provides methods of removing fluid from a subject by administering a cross-linked polyelectrolyte polymer that absorbs about 20-fold or more of its mass in saline and administering an agent that increases the fluid present in the intestine of the subject.

Crosslinked polyelectrolytes designed to act as superabsorbent polymers can be used to provide new methods for the treatment of fluid overload states. One form of crosslinked polyelectrolyte consists of polymers containing many carboxylic acid groups, which can be reacted with alkali metals to produce polycarboxylates. Many of these polycarboxylates act as superabsorbent polymers, absorbing over 20 times their mass in 0.9% saline. Examples of monomer units that can be used to prepare crosslinked polyelectrolytes include, but are not limited to acrylic acid and its salts, methacrylic acid and its salts, crotonic acid and its salts, tiglinic acid and its salts, 2-methyl-2-butenoic acid (Z) and its salts, 3-butenoic acid (vinylacetic acid) and its salts, 1-cyclopentene carboxylic acid, and 2-cyclopentene carboxylic acid and their salts. Other crosslinked polyelectrolyte superabsorbent polymers may be based on sulfonic acids and their salts, phosphonic acids and their salts, or amines and their salts. Those skilled in the art will be able to note that this is not an exhaustive list and is intended for illustrative purposes only.

Polyacrylates, one class of polyelectrolytes, can be formed by copolymerizing an ethylenically unsaturated carboxylic acid with a multifunctional cross-linking monomer. The acid monomer or polymer can be substantially or partially neutralized with an alkali metal salt such as the hydroxide, the carbonate, or the bicarbonate and polymerized by the addition of an initiator. One popular polymer gel is a copolymer of acrylic acid/sodium acrylate and any of a variety of cross-linkers. Although absorbent polymers theoretically could find use in the treatment of fluid-overload diseases such as those described above, to date polymers having sufficient absorption capacity for the remediation of such diseases have not been developed.

An agent capable of preventing or inhibiting absorption of the fluid from the small bowel or colon into the bloodstream to a degree that allows increased swelling of the polyelectrolyte can be co-administered with the polyelectrolyte. Similarly, agents which increase secretion of fluid into the small bowel or colon may be used to increase the fluid in the intestinal tract. Suitable agents include simple osmotic agents such as non-absorbed sugars (mannitol, sorbitol, and the like), small molecular weight, non-absorbed glycols (e.g., polyethylene glycols such PEG 400 to PEG 10,000 and polypropylene glycols), and poorly absorbed salts (magnesium sulfate, sodium sulfate, and the like) so long as they can retain or increase fluid in the intestine. In addition, irritants, such as phenolphthalein, ricinoleic acid, and ispaghula, can also be used. Agents which inhibit the sodium transport in the intestinal wall, such as ouabain or amiloride are also suitable. Agents which stimulate fluid secretion, such as cholera toxin, heat stable toxin from E. coli, toxin from C. difficile, and prostaglandins or hormones such as lubiprostone can also increase the fluid available in the intestine.

Surprisingly, it has been discovered that a superabsorbent polyelectrolyte given to a mammal in combination with certain of these agents that retard or prevent fluid absorption from the intestine or increase secretion of fluid into the intestine are capable of removing more fluid from the mammal via gastrointestinal excretion than the superabsorbent polyelectrolyte alone while other such agents result in less fluid being removed from the mammal by the superabsorbent polyelectrolyte.

Polyelectrolyte materials capable of holding many times their mass in saline, methods for their preparation and use in absorbing fluid in the intestinal lumen are described. Composition comprising such polyelectrolyte materials and materials capable of inhibiting the net absorption of fluid are disclosed. The polyelectrolyte material can be composed of a single class of polymer, such as polyacrylate. The material used to inhibit the net fluid absorption from the intestine can be a single agent or a combination of agents and can be selected from osmotic agents (e.g., non-absorbed, non-fermentable), sodium pump inhibitors, agents increasing fluid secretion and irritants as described above.

In one method, an agent capable of preventing or inhibiting absorption of the fluid from the small bowel or colon into the bloodstream to a degree that allows increased swelling of the polyelectrolyte can be co-administered with the polyelectrolyte. Similarly, agents which increase secretion of fluid into the small bowel or colon may be used to increase the fluid in the intestinal tract.

In some embodiments, the methods may further comprise the step of identifying a subject in need of fluid removal.

In some embodiments, the cross-linked polyelectrolyte polymer is polyacrylate.

In some embodiments, the fluid is physiological saline.

In some embodiments, the agent is selected from the group consisting of: a osmotic agent (e.g., non-fermentable), an inhibitor of intestinal sodium transport and an agent which increases sodium secretion into the intestine. In some embodiments, the agent is selected from the group consisting of: mannitol, polyethylene glycol and lubiprostone. In some embodiments, the polyethylene glycol has a molecular weight between 400 and 10,000 Daltons. In some embodiments, the polyethylene glycol has a molecular weight between 400 and 4000 Daltons. In some embodiments, the agent is a non-absorbed sugar. In some embodiments, the agent is a poorly absorbed salt. In some embodiments, the agent is an irritant. In some embodiments, the agent inhibits sodium transport in the intestinal wall. In some embodiments, the agent stimulates fluid secretion into the intestine.

In some embodiments, the agent is a simple osmotic agent such as a non-absorbed sugar (e.g., mannitol, sorbitol), a small molecular weight, non-absorbed polyethylene glycol (e.g., PEG 400 to PEG 10,000), or a poorly absorbed salt (e.g., magnesium sulfate, sodium sulfate) so long as they can retain fluid in the intestine. In some embodiments, the agent is an irritant, such as phenolphthalein, ricinoleic acid, and ispaghula. In some embodiments, the agent is one which inhibits the sodium transport in the intestinal wall, such as ouabain or amiloride. In some embodiments, the agent is one which stimulates fluid secretion, such as cholera toxin, heat stable toxin from E. coli, toxin from C. difficile, and prostaglandins or hormones such as lubiprostone.

In some embodiments, the cross-linked polyelectrolyte polymer absorbs at least 20-fold, 30-fold, 40-fold, 50-fold, 60-fold, 70-fold, 80-fold, 90-fold, 100-fold or 110-fold or more of its mass in aqueous saline. In some embodiments, the cross-linked polyelectrolyte polymer absorbs more than 60-fold or more of its mass in aqueous saline. In some embodiments, the cross-linked polyelectrolyte polymer absorbs about 70-fold or more of its mass in aqueous saline. In some embodiments, the cross-linked polyelectrolyte polymer absorbs about 80-fold or more of its mass in aqueous saline. In some embodiments, the cross-linked polyelectrolyte polymer absorbs about 90-fold or more of its mass in aqueous saline. In some embodiments, the cross-linked polyelectrolyte polymer absorbs about 100-fold or more of its mass in aqueous saline. In some embodiments, the cross-linked polyelectrolyte polymer absorbs about 110-fold or more of its mass in aqueous saline.

In some embodiments, the cross-linked polyelectrolyte polymer is substantially free of soluble polyacrylic acid polymer.

In some embodiments, the cross-linked polyelectrolyte polymer comprises bound counterions (e.g., cations). In some embodiments, the cross-linked polyelectrolyte polymer comprises one or more bound inorganic counterions. In some embodiments, the inorganic counterion is selected from the group consisting of: hydrogen, sodium, potassium, calcium, magnesium and ammonium. In some embodiments, the cross-linked polyelectrolyte polymer comprises one or more bound organic counterions. In some embodiments, the organic counterion is selected from the group consisting of: choline, arginine and lysine. In some embodiments, the cross-linked polyelectrolyte polymer comprises one or more inorganic counterions and at least one or more organic counterions.

In some embodiments, the cross-linked polyelectrolyte polymer is substantially coated. In some embodiments, the coating is an enteric or delayed release coating.

In some embodiments, the cross-linked polyelectrolyte polymer is substantially in the shape of a disrupted sphere or ellipsoid.

In some embodiments, the cross-linked polyelectrolyte polymer is disrupted by milling or crushing.

In some embodiments, the cross-linked polyelectrolyte polymer is directly administered to the small intestine. In some embodiments, the cross-linked polyelectrolyte polymer is directly administered to the jejunum. In some embodiments, the cross-linked polyelectrolyte polymer is directly administered to the colon.

In some embodiments, the cross-linked polyelectrolyte polymer is administered orally.

In some embodiments, the subject has cardiac disease. In some embodiments, the cardiac disease is congestive heart failure. In some embodiments, the subject has kidney disease. In some embodiments, the kidney disease is nephrosis, nephritis, chronic kidney disease (CKD), or end stage renal disease (ESRD).

### Preparation of Superabsorbent Polyelectrolyte Beads

Superabsorbent polyelectrolyte beads, including, for example, polyacrylate beads, may be prepared by methods known in the art, including by suspension methods (e.g., Buchholz, F. L. and Graham, A. T., “Modern Superabsorbent Polymer Technology,” John Wiley & Sons (1998)). Methods may include manufacture of polyelectrolyte beads by inverse suspension polymerization. Exemplary methods are provided below.

1. Manufacture of Superabsorbent Polyelectrolyte

Cross-linked polyelectrolyte polymers, including cross-linked polyelectrolyte polymeric beads, may be prepared by commonly known methods in the art. In an exemplary method, cross-linked polyelectrolyte polymers may be prepared as a suspension of drops of aqueous solution in a hydrocarbon (e.g., by inverse suspension polymerization).

Superabsorbent polyacrylates may be prepared by polymerization of partially neutralized acrylic acid in an aqueous environment where an appropriate cross-linker is present in small quantities. Given that there is an inverse relationship between the amount of fluid the superabsorbent polymer will absorb and the degree of cross-linking of the polymer, it desirable to have the minimum cross-linking possible to still produce a resin. However, there is also an inverse relationship between the degree of cross-linking and the percentage of polymer chains that do not cross-link and are therefore soluble polymer that does not contribute to the absorbency of the resin since it dissolves in the fluid. For example, superabsorbent polyacrylates can be designed to absorb about 35 times their mass in physiological saline as a compromise between maximal absorbency and minimal soluble polymer.

Since the amount of reactants used in an inverse suspension polymerization reaction varies depending upon the size of the reactor, the precise amount of each reactant used in the preparation of cross-linked polyelectrolyte polymer, such as polyacrylate may be determined by one of skill in the art. For example, in a five-hundred gallon reactor, about 190 to 200 pounds (roughly 85 to 90 kg) of acrylic acid may be used while in a three liter reactor 150 to 180 g of acrylic acid may be used. Accordingly, the amounts of each reactant used for the preparation of cross-linked polyacrylate are expressed as weight ratios to acrylic acid. Thus, acrylic acid weight is taken as 1.0000 and other compounds are presented in relation to this value. Exemplary amounts of reactants used for the preparation of cross-linked polyacrylate by an inverse suspension polymerization are presented in Table 1.

TABLE 1
Exemplary amounts of reactants in an
inverse suspension polymerization

| Substance | Low value | High Value |
|---|---|---|
| Acrylic acid | 1.0000 | 1.0000 |
| Water | 0.5000 | 3.0000 |
| Hydrophobic solvent | 1.20000 | 12.0000 |
| Base | 0.6600 | 1.1100 |
| (expressed as 50% NaOH) | (60% neutral) | (100% neutralized) |
| Crosslinker | 0.0030 | 0.0080 |
| Initiator | 0.0005 | 0.0200 |
| Chelating agent | 0.0000 | 0.0050 |
| Surfactant | 0.0050 | 0.0400 |

An exemplary inverse suspension reaction to form a superabsorbent polymer may involve preparation of two mixtures (e.g., a hydrophobic and an aqueous mixture) in two different vessels followed by combination of the mixtures to form a reaction mixture. One vessel may be designated as a hydrophobic compound vessel and the other may be designated as a aqueous solution vessel. The hydrophobic compounds may be mixed in a larger vessel that will become a reaction vessel, while an aqueous solution may be prepared in a smaller vessel that may be discharged into the reaction vessel.

A hydrophobic solvent may be introduced into the reaction vessel. As will be appreciated by one of skill in the art, a hydrophobic solvent (also referred to herein as the “oil phase”) may be chosen based upon one or more considerations, including, for example, the density and viscosity of the oil phase, the solubility of water in the oil phase, the partitioning of the neutralized and unneutralized ethylenically unsaturated monomers between the oil phase and the aqueous phase, the partitioning of the crosslinker and the initiator between the oil phase and the aqueous phase and/or the boiling point of the oil phase.

Hydrophobic solvents contemplated for use in the present disclosure include, for example, Isopar L, toluene, benzene, dodecane, cyclohexane, n-heptane and/or cumene. Preferably, Isopar L is chosen as a hydrophobic solvent due to its low viscosity, high boiling point and low solubility for neutralized monomers such as sodium acrylate and/or potassium acrylate. One of skill in the art will appreciate that a large enough volume of hydrophobic solvent is used to ensure that the aqueous phase is suspended as droplets in the oil rather than the reverse and that the aqueous phase droplets are sufficiently separated to prevent coalescence into large masses of aqueous phase.

One or more surfactants and one or more crosslinkers may be added to the oil phase. The oil phase may then be agitated and sparged with an inert gas, such as nitrogen or argon to remove oxygen from the oil phase. It will be appreciated that the amount of surfactant used in the reaction depends on the size of the desired beads and the agitator stir rate. This addition of surfactant is designed to coat the water droplets formed in the initial reaction mixture before the reaction starts. Higher amounts of surfactant and higher agitation rates produce smaller droplets with more total surface area. It will be understood by those of skill in the art that an appropriate choice of cross-linker and initiator may be used to prepare spherical to ellipsoid shaped beads. One of skill in the art will be capable of determining an appropriate cross-linker for the preparation of a specified cross-linked polyelectrolyte. For example, cross-linker choice depends on whether it needs to be hydrophobic or hydrophilic or whether it needs to resist acidic or basic external conditions. An amount of cross-linker depends on how much soluble polymer is permissible and how much saline holding capacity is needed.

Exemplary surfactants include hydrophobic agents that are solids at room temperature, including, for example, hydrophobic silicas (such as Aerosil or Perform-O-Sil) and glycolipids (such as polyethylene glycol distearate, polyethylene glycol dioleate, sorbitan monostearate, sorbitan monooleate or ocytl glucoside).

Crosslinking agents with two or more vinyl groups that are not in resonance with each other may be used, allowing for a wide variety in molecular weight, aqueous solubility and/or lipid solubility. Crosslinking agents contemplated for use in the present disclosure, include, for example, diethyleneglycol diacrylate (diacryl glycerol), triallylamine, tetraallyloxyethane, allylmethacrylate, 1,1,1-trimethylolpropane triacrylate (TMPTA), and divinylbenzene.

An aqueous phase mixture may be prepared in another vessel (e.g., a vessel that is separate from that used to prepare the hydrophobic phase) by placing water into the vessel and adding a base to the water. It will be appreciated by one of skill in the art that the amount of base used in the vessel is determined by the degree of neutralization of the monomer desired. A degree of neutralization between 60% and 100% is preferred. Without wishing to be bound by a theory of the disclosure, it is believed that one-hundred percent neutralization minimizes the chance of suspension failure, but the highly charged monomer may not react as rapidly and may not pull hydrophobic crosslinkers into the beads. Considerations in choosing the degree of neutralization may be determined by one of skill in the art and include, for example, the effect of monomer charge (e.g., as determined by ionization of the cation from the neutralized molecules) on reaction rate, partitioning of the monomer and neutralized monomer between oil phase and aqueous phase and/or tendency to coalescence of the polymer chains during the reaction. The solubilities of sodium acrylate and sodium methacrylate in water are limited and are lower at lower temperatures (e.g., sodium acrylate is soluble at about 45% at 70° C. but less than 40% at 20° C.). This solubility may establish the lower limit of the amount of water needed in the neutralization step. The upper limit of the amount of water may be based on reactor size, amount of oil phase needed to reliably suspend the aqueous phase as droplets and/or the desired amount of polymer produced per batch.

Bases contemplated for use in the present disclosure include, for example, hydroxides, bicarbonates, or carbonates. Use of these bases allows neutralization of the acid monomer without residual anions left in the reaction mixture. It will be apparent to one of skill in the art that the cation used for the base may be chosen based on the planned use of the superabsorbent polymer. Normally, sodium bases are chosen since the superabsorbent polymers will be used in situations where saline solutions will be encountered. However, potassium bases, ammonium bases, and bases of other cations are contemplated for use in the present disclosure.

The water used in the reaction may be purified water or water from other sources such as city water or well water. If the water used is not purified water, chelating agents may be needed to control metals such as iron, calcium, and magnesium from destroying the initiator. Chelating agents contemplated for use with the present disclosure include, for example, Versenex 80. The amount of chelating agent added to the reaction mixture may be determined by one of skill in the art from a determination of the amount of metal in the water.

Once base is added to the water, the aqueous phase solution may be cooled to remove the heat released from dilution of the base and one or more classes of monomers may be added to react with the base. As will be appreciated by one of skill in the art, the monomers will be neutralized to the degree dictated by the amount of base in the reaction. The aqueous phase solution may be kept cool (e.g., below 35 to 40° C.) and preferably around 20° C. to prevent formation of prepolymer strands, dimers and/or possible premature polymerization.

Monomers are dissolved in water at concentrations of 20-40 wt % and polymerization may subsequently be initiated by free radicals in the aqueous phase. Monomers may be polymerized either in the acid form (pH 2-4) or as a partially neutralized salt (pH 5-7). The amount of water used to dissolve the monomer is minimally set so that all of the monomer (e.g., sodium acrylate) is dissolved in the water rather than crystallizing and maximally set so that there is the smallest volume of reaction mixture possible (to minimize the amount of distillation and allow the maximum yield per batch).

Exemplary monomer units contemplated for use in the present disclosure, include, for example, acrylic acid and its salts, methacrylic acid and its salts, crotonic acid and its salts, tiglinic acid and its salts, 2-methyl-2-butenoic acid and its salts, 3-butenoic acid (vinylacetic acid) and its salts, 1-cyclopentene carboxylic acid, and 2-cyclopentene carboxylic acid and their salts. Other cross-linked polyelectrolyte superabsorbent polymers may be based on sulfonic acids and their salts, phosphonic acids and their salts, or amines and their salts.

One or more initiators, free radical producers, may be added to the aqueous phase just before the aqueous phase is transferred into the oil phase. As will be appreciated by one of skill in the art, the initiator amounts and type used in the polymerization reaction depend on oil versus water solubility and the need for longer chain lengths. For example, a lower amount of initiator may be used in the polymerization reaction when longer chain lengths are desired.

In some embodiments, the initiator may be a thermally sensitive compound such as persulfates, 2,2′-azobis(2-amidino-propane)-dihydrochloride, 2,2′-azobis(2-amidino-propane)-dihydrochloride and/or 2,2′-azobis(4-cyanopentanoic acid) persulfate or 2,2′-azobis(4-cyanopentanoic acid). Thermally sensitive initiators have the disadvantage that the polymerization does not begin until an elevated temperature is reached. For persulfates, this temperature is approximately 50 to 55° C. Since the reaction is highly exothermic, vigorous removal of the heat of reaction is required to prevent boiling of the aqueous phase. It is preferred that the reaction mixture be maintained at approximately 65° C. As will be appreciated by one of skill in the art, thermal initiators have the advantage of allowing control of the start of the reaction when the reaction mixture is adequately sparged of oxygen.

In some embodiments, the initiator may also be a redox pair such as persulfate/bisulfate, persulfate/thiosulfate, persulfate/ascorbate, hydrogen peroxide/ascorbate, sulfur dioxide/tert-butylhydroperoxide, persulfate/erythorbate, tert-butylhydroperoxide/erythorbate and/or tert-butylperbenzoate/erythorbate. These initiators are able to initiate the reaction at room temperature, thereby minimizing the chance of heating the reaction mixture to the boiling point of the aqueous phase as heat is removed through the jacket around the reactor. However, homogeneous mixing may not accomplished by the time the reaction is initiated and there may be rapid polymerization of the surface of the droplets with much slower polymerization within the bead.

In preferred embodiments, the reaction is not started immediately after the mixing of the aqueous phase into the oil phase in the final reactor because the aqueous phase still has an excessive amount of oxygen dissolved in the water. It will be appreciated by one of skill in the art that an excessive amount of oxygen cause poor reactivity and inadequate mixing may prevent the establishment of uniform droplet sizes. Instead, the final reaction mixture is first sparged with the inert gas for ten to sixty minutes after all reagents (except the redox pair if that initiator system is used) have been placed in the reactor. The reaction may be initiated when a low oxygen content (e.g., below 15 ppm) is measured in the inert gas exiting the reactor.

It will be appreciated by those of skill in the art that with acrylate and methacrylate monomers polymerization begins in the droplets and progresses to a point where coalescence of the beads becomes more likely (the “sticky phase”). It may be necessary that a second addition of surfactant (e.g., appropriately degassed to remove oxygen) be added during this phase or that the agitation rate be increased. For persulfate thermal initiation, this sticky phase may occur at about 50 to 55° C. For redox initiation systems, the need for additional surfactant may be lessened by the initial surface polymerization, but if additional surfactant is needed, it should be added as soon as an exotherm is noted.

The reaction may be continued for four to six hours after the peak exotherm is seen to allow for maximal consumption of the monomer into the polymer. Following the reaction, the beads may be isolated by either transferring the entire reaction mixture to a centrifuge or filter to remove the fluids or by initially distilling the water and some of the oil phase (e.g., frequently as an azeotrope) until no further removal of water is possible and the distillation temperature rises significantly above 100° C. followed by isolating the beads by either centrifugation or filtering. The isolated beads are then dried to a desired residual moisture content (e.g., less than 5%).

An exemplary cross-linked polyelectrolyte, polyacrlylate, may be formed by copolymerizing an ethylenically unsaturated carboxylic acid with a multifunctional cross-linking monomer. The acid monomer or polymer may be substantially or partially neutralized with an alkali metal salt such as the hydroxide, the carbonate, or the bicarbonate and polymerized by the addition of an initiator. One such exemplary polymer gel is a copolymer of acrylic acid/sodium acrylate and any of a variety of cross-linkers.

The reactants for the synthesis of exemplary cross-linked polyelectrolyte polymeric beads, such as cross-linked polyacrylate, are provided in Table 2 below. These cross-linked polyelectrolyte polymeric beads may be produced as a one-hundred kilogram batch in a five-hundred gallon vessel.

TABLE 2
List of Components Used in the Manufacture
of Cross-linked Polyacrylate Beads

| | | Amount/batch |
|---|---|---|
| Component | Function | (kg) |
| Acrylic Acid | Monomer | 88 |
| Water | Solvent | 90 |
| 50% Sodium Hydroxide | Neutralization of acrylic | 79 |
| | acid monomer | |
| Naphtha [petroleum], | Continuous phase for | As needed |
| hydrotreated heavy, (Isopar L) | suspension | |
| Fumed silica (Aerosil R972) | Suspending agent | 0.9 |
| Diethylenetriaminepentaacetic | Control of metal ions in | 0.9 |
| Acid Pentasodium | reagents, solvents, or | |
| Sodium Persulfate | Polymerization initiator | 0.06 |
| Trimethylolpropane | Cross-linking agent | 0.3 |
| Triacrylate, (TMPTA) | | |

An exemplary polymerization reaction is shown below.

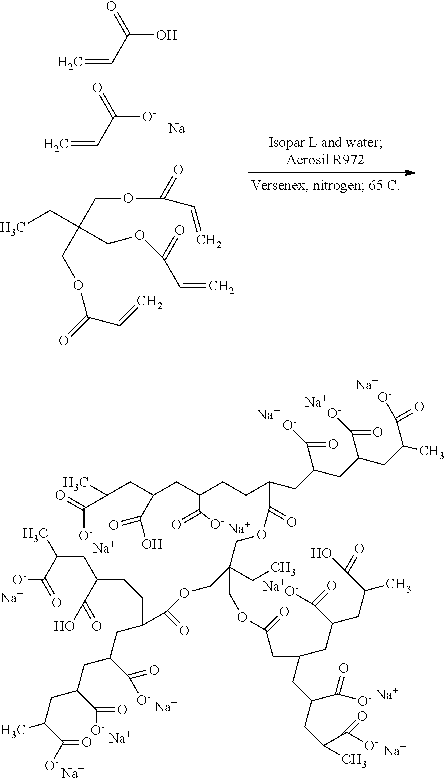

2. Preparation of Cross-Linked Polyelectrolyte Polymeric Beads with Hydrogen Counterions

Partially neutralized or non-neutralized polyelectrolyte polymers may be prepared with 100% hydrogen counterion content by washing the polymer with acid. Suitable acids contemplated for use with the present disclosure, include, for example, hydrochloric acid, acetic acid and phosphoric acid.

Those skilled in the art will recognize that the replacement of the counterions, including cations such as sodium atoms, by hydrogen atoms can be performed with many different acids and different concentrations of acid. However, care must be taken in choice of acid and concentration to avoid damage to the polymer or the cross-linkers. For instance, nitric and sulfuric acids would be avoided.

Acid washed polyelectrolyte polymers may then be dried in a vacuum oven or inert atmosphere until less than 5% moisture remains to produce cross-linked polyacrylic acid which is substantially the free acid form of lightly cross-linked polyacrylic acid. Optionally, if the intact bead form of partially-neutralized, lightly cross-linked polyacrylate is used, the cross-linked polyelectrolyte polymer may be left in the bead form recovered from the oven or may be milled to obtain smaller particles of low-sodium cross-linked polyelectrolyte polymer.

3. Preparation of Cross-Linked Polyelectrolyte Polymeric Beads with Varying Counterion Content

The free acid form of cross-linked polyelectrolyte polymers of the present disclosure, including, for example, cross-linked polyacrylic acid may be converted into polymer with various levels of one or more counterions (e.g., one or more inorganic counterions, such as sodium, potassium, calcium, magnesium and/or ammonium and/or one or more organic counterions, such as choline and/or lysine). These methods may be carried out with intact beads, with disrupted beads, or with powdered forms of cross-linked polyelectrolyte polymers, including for example, polyacrylate polymers.

Suitable counterions include alkali metals and alkaline earth metals, including, for example, sodium, potassium, calcium or magnesium and exclude hydrogen. Counterions may be selected based on the requirements of an individual subject. For example, by appropriate selection of counterions electrolytic imbalances in subjects may be treated. For example, in subjects having excess sodium, sodium would be avoided as a counterion.

Counterions may be provided as salts that could be dissolved to a sufficient degree in aqueous solution and mixed with the acid form of the polymer. Particularly advantageous choices of salts would be those that neutralize the acid in such a way as to produce products that are easily removed from the polymer. Such salts include the carbonate salt of the desired counterion (e.g. sodium carbonate, potassium carbonate, calcium carbonate), the bicarbonate salt of the desired counterion (e.g. calcium bicarbonate, magnesium bicarbonate, lithium bicarbonate), or the hydroxide or oxide of the desired counterion (e.g. sodium hydroxide, choline hydroxide, magnesium hydroxide, magnesium oxide).

4. Preparation of Cross-Linked Polyelectrolyte Polymeric Beads with Increased Saline Holding Capacity

Partially neutralized or non-neutralized polyelectrolyte polymers of the present disclosure, including cross-linked polyelectrolyte polymeric beads, may be disrupted to increase their saline holding capacity. Saline holding capacity is preferably determined as described in Example 4, wherein the beads or disrupted beads are include with a neutral pH (e.g., pH 7) saline solution having a sodium concentration of 0.15 M. Alternatively, a 0.9% saline solution (0.154 M sodium) may be used.

Cross-linked polyelectrolyte polymeric beads, including cross-linked polyacrylate polymeric beads, may be disrupted into smaller particles, for example, by milling or crushing in a grinder. The disrupted polymeric beads are preferably washed to remove soluble polymer. Suitable washing solutions include purified water such as deionized water or distilled water and various alcohols. Since the polymer is to be dried, it is desirable to use fluids that will evaporate easily without leaving any residue, such as salts, in the dried polymer. Alternatively, cross-linked polyelectrolyte polymeric beads, including cross-linked polyacrylate polymeric beads may be disrupted by placing the beads into purified water and agitating the beads (e.g., stirring with a magnetic stir bar or agitating at 500 rpm overnight), the residual soluble polymer in the polymeric beads may be reduced or eliminated and the saline holding capacity of the polymeric beads increased.

Particles of a certain size, may be obtained by sieving through sieves such as screens. Screens may be stacked to obtain particles with a range of sizes. Screens are shaken to allow particles to sift through and get caught on the screen with an opening just below their diameter. For example, particles that pass through an 18 Mesh screen and are caught on a 20 Mesh screen are between 850 and 1000 microns in diameter. Screen mesh and the corresponding particle size allowed to pass through the mesh include, 18 mesh, 1000 microns; 20 mesh, 850 microns; 25 mesh, 710 microns; 30 mesh, 600 microns; 35 mesh, 500 microns, 40 mesh, 425 microns; 45 mesh, 35 microns; 50 mesh, 300 microns; 60 mesh, 250 microns; 70 mesh, 212 microns; 80 mesh, 180 microns; 100 mesh, 150 microns; 120 mesh, 125 microns; 140 mesh, 106 microns; 170 mesh, 90 microns; 200 mesh, 75 microns; 230 mesh, 63 microns; and 270 mesh, 53 microns. Thus particles of varying sizes may be obtained through the use of one or more screens.

### Therapeutic Uses

The disclosed polymers and agents that increase fluid in the intestine (e.g., osmotic agents, irritants, sodium absorption blocking agents and agents that enhance fluid secretion) have a variety of uses, including therapeutic uses. Such methods may include removal of fluid. Such methods may also include treating diseases or disorders associated with increased retention of fluid and/or ion imbalances. The disclosed polymers may be used in methods to treat end stage renal disease (ESRD), chronic kidney disease (CKD), congestive heart failure (CHF) or hypertension. The disclosed polymers may also be used in methods to treat an intestinal disorder, a nutritional disorder (e.g., kwashiorkor or gluten-sensitive enteropathy), a hepatic disease (e.g., cirrhosis of the liver), an endocrine disorder (e.g., preclampsia or eclampsia), a neurological disorder (e.g., angioneurotic edema) or immune system disorder.

In some embodiments, the absorbent material may be encapsulated in a capsule. The capsules may be coated with a coating that allows it to pass through the gut and open in the intestine where the material may absorb fluid or specific ions that are concentrated in that particular position of the intestine. The individual particles or groups of particles may be encapsulated or alternatively, larger quantities of beads or particles may be encapsulated together.

In an exemplary method, the swelling rate of the polymer may be controlled by selecting particle or bead size, and or polymer with varied level of ion loading, to provide delivery of the polymer to specific locations in the gut before extensive swelling occurs. Larger sized particles have slower swelling rates. When given orally, the absorbent material may be used to supplement or replace dialysis treatments in dialysis patients, to supplement or replace diuretic therapy in patients with congestive heart failure, to supplement or replace diuretic and antihypertensive therapy in patients with hypertension and to supplement or replace these and dietary measures for treatment of fluid and/or sodium overload and/or potassium overload in patients with other diseases and syndromes, including those causing fluid retention in the body.

The methods may be used to modulate (e.g., increase or decrease) levels of one or more ions, including more than one ion, in a subject by administering a composition of the present disclosure to the subject in an amount effective to modulate the levels of one or more ions, including more than one ion, in the subject.

The composition may bind to one or more ions in the subject thereby decreasing the levels of one or more ions in the subject. Additionally, the composition may release one or more ions in the subject thereby increasing the levels of one or more ions in the subject. Alternatively, the composition may bind to one or more first ions in the subject thereby decreasing the levels of one or more first ions in the subject and the composition release one or more second ions in the subject thereby increasing the levels of one or more second ions in the subject.

The composition may be used to remove one or more ions selected from the group consisting of: hydrogen, sodium, potassium, calcium, magnesium and/or ammonium.

### Pharmaceutical Compositions

Pharmaceutical compositions are disclosed comprising a cross-linked polyelectrolyte polymer, including cross-linked polyelectrolyte polymeric beads, of the present disclosure. These compositions may be delivered to a subject, including a subject using a wide variety of routes or modes of administration. Preferred routes for administration are oral or intestinal.

A pharmaceutical composition or dosage form, including wherein the polymer is in admixture or mixture with one or more pharmaceutically acceptable carriers, excipients or diluents. Pharmaceutical compositions for use in accordance with the present disclosure may be formulated in conventional manner using one or more physiologically acceptable carriers compromising excipients and auxiliaries which facilitate processing of the polymer into preparations which may be used pharmaceutically. Proper formulation is dependent upon the route of administration chosen. Such compositions may contain a therapeutically effective amount of polymer and may include a pharmaceutically acceptable carrier. Pharmaceutically acceptable carriers include those approved by a regulatory agency of the Federal or a state government or listed in the U.S. Pharmacopeia or other generally recognized pharmacopeia for use in animals, and more particularly, in humans. Carriers can include an active ingredient in which the disclosed compositions are administered.

For oral administration, the disclosed compositions may be formulated readily by combining them with pharmaceutically acceptable carriers well known in the art. Such carriers enable the compositions of the disclosure to be formulated, preferably in capsules but alternatively in other dosage forms such as tablets, pills, dragees, capsules, liquids, gels, syrups, slurries, suspensions, wafers, sachets, powders, dissolving tablets and the like, for oral ingestion by a subject, including a subject to be treated. In some embodiments, the compositions or capsules containing the compositions, do not have an enteric coating.

The amount of the active cross-linked polyelectrolyte polymer, including cross-linked polyelectrolyte polymeric beads, are present in an effective amount, including, for example, in an amount effective to achieve therapeutic and/or prophylactic benefit. Effective doses may be extrapolated from dose-response curves derived from in vitro or animal model test systems. Dosage amount and interval may be adjusted individually to provide levels of cross-linked polyelectrolyte polymer, including cross-linked polyelectrolyte polymeric beads that are sufficient to maintain the desired therapeutic effect. The dosage regimen involved in a method of treatment may be determined by the attending physician, considering various factors which modify the action of polymer, e.g. the age, condition, body weight, sex and diet of the subject, the severity of disease, time of administration and other clinical factors.

The amount of compound administered will, of course, be dependent on the subject being treated, on the subject's weight, the nature and severity of the affliction, the manner of administration, and the judgment of the prescribing physician. The therapy may be repeated intermittently while symptoms are detectable or even when they are not detectable. The therapy may be provided alone or in combination with other agents.

As described above, the polyelectrolyte polymer of the present disclosure is administered in combination with an agent that increases the amount of fluid in the intestine. For example, to enhance the ability of the polymer (e.g., superabsorbent polyelectrolyte) to absorb fluid from the gastrointestinal tract (e.g., small intestine), the addition of an agent designed to increase or retain fluid in the intestine by either osmotically pulling fluid from the bloodstream into the intestine, osmotically inhibiting the absorption of fluid from the intestine, inhibiting the sodium absorption (and the fluid which migrates to maintain isotonicity) from the intestine, or increasing the secretion of ions (such as sodium or chloride, along with the water needed to retain isotonicity) into the intestine is given in combination with the cross-linked polyelectrolyte polymer. An effective dose of such an agent may be determined clinically. Other therapeutic agents may also be administered depending in part, on the condition being treated.

### EXAMPLES

### Example 1

This example demonstrates the preparation of an exemplary cross-linked polyelectrolyte polymer, such as a lightly crosslinked polyacrylic acid partially neutralized with sodium.

An inverse suspension process may be used with the following components: a monomer (e.g., polyacrylic acid), solvent (e.g., water), base for neutralization of monomer (e.g., NaOH), lipophilic solvent (e.g., Isopar L), suspending agent (e.g., fumed silica such as Aerosil R972), chelating agent (e.g., Versenex-80), polymerization initiator (e.g., sodium persulfate), and cross-linking agent (e.g., TMPTA). For example, cross-linked polyacrylate beads were prepared by adding eighty-eight kilograms acrylic acid and about eighty-seven kilograms of water to a suitable, agitated vessel and sparging air through the mixture. The mixture was continuously agitated and cooled while seventy-nine kilograms of 50% sodium hydroxide was added while the temperature of the mixture was advantageously maintained below about 40° C. In this manner about 80% neutralization of the acrylic acid was obtained. If desired, neutralization percentages of from about 60% to 100% were obtained by altering the amount of sodium hydroxide. Alternatively other basic sodium salts, such as sodium carbonate or sodium bicarbonate, are used in addition to basic salts of other alkali metals.

To a second, suitable, agitated reactor, about seven-hundred kilograms of Isopar L (or other lipophilic solvents such as toluene, heptane, cyclohexane) was added to 0.3 kilograms of fumed silica (Aerosil R972) that is pre-dispersed in about twenty kilograms of Isopar L (or other lipophilic solvent). Next, about 0.9 kilograms of Versenex-80 solution was added to the partially neutralized acrylic acid solution followed by the addition of 0.3 kilograms of trimethylolpropane triacrylate to the Isopar L/Aerosil R972 dispersion. About 0.06 kilograms sodium persulfate as a solution in about three kilograms of water was added to the partially neutralized acrylic acid solution. The partially neutralized acrylic acid solution may then be filtered.

The partially neutralized acrylic acid solution was transferred into the Isopar L in the second reactor. Optionally, the partially neutralized acrylic acid solution may be filtered at this point. The mixture was agitated for about fifteen to thirty minutes to achieve suspension of the aqueous monomer droplets while nitrogen (or other suitable inert gas) was sparged through the mixture during the agitation period. The reactor temperature may be increased to about 50° C. at which point a second dispersion of Aerosil R972 (0.6 kilograms of Aerosil R972 in about twenty kilograms of Isopar L) may be added to the reaction mixture. Polymerization of the mixture was completed by heating the reaction mixture to about 65° C. and holding the contents at about 65° C. for about two to four hours after the peak exotherm was observed. The reactor contents were then cooled and placed under vacuum to remove water. About two-hundred and twenty kilograms of distillate was collected. The beads were isolated by centrifugation and dried under vacuum with a nitrogen bleed, if needed, at about 100° C.

The beads were screened to remove oversized agglomerates and fines. Typically, about one-hundred kilograms of cross-linked polyacrylate beads were obtained. If the residual acrylic acid level is too high, the cross-linked polyacrylate beads are reloaded to a suitable reactor containing Isopar L, water, and a small amount of sodium persulfate. After sparging the mixture with nitrogen, the beads were incubated at about 70° C. for about two to three hours. The mixture was then cooled and the cross-linked polyacrylate beads isolated, dried, and screened as before.

When the beads were screened, the mean particle size for the beads generally ranged from about 700 microns to about 1200 microns. The upper screen size ranged from 840 to 1400 microns (e.g., 24-16 mesh) and the lower screen size ranged from 540 to 840 microns (e.g., 36-24 mesh).

Optionally, the beads are placed into capsules (e.g., hard size 00 HPMC capsules). Such capsules are optionally coated. The following materials are used to prepare an exemplary coating suspension (% w/w): Eudragite L30D-55 (53.76%), Plasacryl (6.45%), triethyl citrate (2.58%) and sterile water (37.20%). For example, L30D-55 is dispensed into a steel container with agitation to create a vortex. Next, sterile water, Plasacryl and triethyl citrate are added to the vortex. The capsules may then be sprayed with the mixture followed by drying.

### Example 2

This example demonstrates the preparation of an exemplary cross-linked polyelectrolyte polymer, such as a cross-linked polyacrylate polymer.

Cross-linked polyelectrolyte was prepared on a smaller scale by placing 14.7 kg Isopar L (or other inert hydrocarbon solvent such as toluene, cyclohexane, or n-heptane) into a jacketed, thirty liter glass or stainless steel reactor fitted with two low-shear, high-viscosity impellers and two baffles. 0.0086 kg of fumed silica, such as, Aerosil R972 and 0.5 kg of Isopar L (or whichever hydrocarbon solvent has been chosen) were added to the high shear blender such as a Waring blender to disperse the Aerosil into the solvent for two minutes. Next, the mixture was added to the thirty liter reactor. The solution was then agitated in the thirty liter reactor while an inert gas was sparged through the room temperature solution.

A second batch of 0.5 kg Isopar L (or whichever hydrocarbon solvent has been chosen) with 0.0086 kg Aerosil R972 was prepared in a high shear blender. This suspension was placed into a vessel and an inert gas (nitrogen, argon, etc) sparged through it to degas it. The degassing was continued until the solution was used.

About 1.72 kg glacial acrylic acid and 1.72 kg water was placed into a twelve liter jacketed reactor and the temperature lowered to about 15° C. With vigorous stirring, 1.53 kg of 50% NaOH solution was added while keeping the temperature below 30° C. Air was maintained in the reaction mixture by bubbling through the solution, if needed. When the neutralization addition was completed, 0.069 kg of 10% Versenex 80 solution was added to the reactor and mixed. After a few minutes, 0.009 kg of freshly prepared 10% sodium persulfate solution was added to the reactor and mixed for a few minutes. The solution was then transferred to the thirty liter reactor.

About 0.006 kg of trimethylolpropane was added to the thirty liter reactor. The agitation was continued in the thirty liter reactor while de-gassing by bubbling an inert gas through the mixture for 40 to 60 minutes. The solution was kept at room temperature. After the 40 to 60 minutes of degassing, the temperature of the reaction mixture was quickly raised by circulation of a 90 to 95° C. solution through the jacket of the jacketed reactor while continuing the degassing and agitation. When the reaction mixture reaches 50° C., the second batch of Aerosil R972 was rapidly added. When the reaction mixture reaches 60° C., the temperature of the heating bath was reduced to 65° C. and the reaction mixture maintained at 65° C. for 2 to 4 hours.

After two to four hours, the reaction mixture was distilled under partial vacuum until no water was being removed and the reaction mixture was cooled to room temperature. The beads were filtered from the liquid and dried under an inert atmosphere until less than 5% moisture remains. Alternatively, the beads are isolated by filtration immediately after the two to four hours of reaction time, rinsed with the organic solvent, and dried under an inert atmosphere. These beads were then processed in the same manners mentioned above to disrupt the beads and wash with purified water to produce the high saline holding capacity CLP described.

### Example 3

This example demonstrates the preparation of an exemplary cross-linked polyelectrolyte polymer, such as a cross-linked polyacrylate polymer.

The bead form of lightly cross-linked, 80% neutralized polyacrylic acid were prepared in a 500 gallon reactor by loading 1775.5 pounds of Isopar L into the reactor and adding 0.4 pounds of Aerosil R972 which had been mixed with high shear in 50.5 pounds of Isopar L. Agitation and nitrogen purge at 500 scfh was started. In a separate reactor, 195.3 pounds of acrylic acid was mixed with 20.7 pounds of water and sparged with air. 176.5 pounds of 50% NaOH solution were added to the acrylic acid over 1.25 hours while the temperature was maintained below 40° C. To this solution, 2.0 pounds of Versenex 80 solution, 0.71 pounds trimethylolpropane triacrylate, and 0.158 pounds of sodium persulfate were added. This solution was then transferred to the primary reactor with continued sparging. A second Aerosil charge was prepared using 1.3 pounds of Aerosil in 50.9 pounds of Isopar L with high shear agitation. After approximately 1 hour of sparging, the reactor was heated to a maximum of 78° C. and held in the heated state for 4 to 5 hours. The reactor was then placed under vacuum and distillation was performed for about 5 hours. The remaining reaction mixture was transferred to a centrifuge where the beads were separated and moved to a drier. The dried beads were sieved to select for beads between 710 microns and 1000 microns.

### Example 4

This example describes an exemplary method for determining saline holding capacity of a cross-linked polyelectrolyte polymer, such as a cross-linked polyacrylate polymer.

A pH seven buffer of sodium phosphate tribasic (Na₃PO₄.12H₂0; MW 380.124) was prepared by dissolving 19.0062 grams in about 950 milliliters pure water and adjusting the pH to a final pH of seven±0.1 with 1N HCl before final dilution to one liter resulting in a solution with a sodium concentration of 0.15 M. Next, an amount of cross-linked polyelectrolyte, for example, cross-linked polyacrylate beads (e.g., 0.2±0.05 grams), were transferred to a tared tube and the mass of the beads recorded as in W1. Next, the tube was returned to the balance to record the weight of the tube plus the sample as W2. An excess (e.g., more than seventy times the mass of polymer) amount of the pH 7.0 buffer (e.g., ten milliliters) was then transferred to the tube containing the CLP sample. The tube was then placed on a flat bed shaker with shaking for two, four or six hours. When reduced sodium cross-linked polyacrylate polymer was being tested for saline holding capacity, this time may be extended to twenty-four hours. After shaking, all excess fluid was removed from the tube (e.g., no visible fluid in the tube). Last, the tube and sample were weighed and recorded as W3. The saline holding capacity (SHC) was calculated by dividing the mass of the dry cross-linked polyacrylate beads into the mass of the fluid absorbed, for example, SHC (g/g)=(W3−W2)/(W1). According to the present disclosure, cross-linked polyelectrolyte polymeric beads, including polyacrylate beads prepared as described in Example 1, have a saline holding capacity of twenty grams per gram, forty grams per gram or more. Alternatively stated, such cross-linked polyelectrolyte polymeric beads, including where the polyelectrolyte is polyacrylate, may absorb 20-fold, 40-fold, or more of their mass in a saline solution.

### Example 5

This example demonstrates that a non-absorbed, non-fermented carbohydrate, such as mannitol, is capable of increasing the fluid absorbed by a superabsorbent polyelectrolyte.

In an exemplary method, rats were placed under isoflurane anesthesia and a midline abdominal incision was performed and two segments of the small intestine of the rat were isolated and closed proximally and distally while keeping the blood supply intact. The segments chosen were the proximal jejunum from the ligament of Trietz to a point approximately four centimeters distal and a segment of four centimeters just proximal to the ileocecal valve. Either cross-linked polyelectrolyte polymer (CLP) alone prepared as described in Example 1 or CLP plus the same weight of mannitol or CLP plus twice that weight of mannitol were placed into each segment, the bowel was returned to the abdomen which was then closed for thirty minutes to allow fluid accumulation in the CLP. Each rat was then sacrificed the rat and the segments are opened to permit weighing of the contents of the segment of intestine. This was done in three or four rats for each treatment—CLP alone, CLP with a 1× dose of mannitol, and CLP with a 2× dose of mannitol. The resulting weights were normalized to give the weight of fluid per gram of CLP. Mannitol doubled the amount of fluid absorbed by the CLP in the jejunal as shown in Table 1. Results obtained with mannitol in the ileum are shown in Table 2. These results suggest that the osmotic agent mannitol increases the fluid retention by a polyelectrolyte in isolated small intestinal segments (e.g. the jejunum), where fermentation does not occur.

TABLE 1
Fluid Accumulation in CLP in Jejunum

| Dose | Mean | Standard Deviation |
|---|---|---|
| CLP | 11.1 | 2.7 |
| CLP, 1X mannitol | 21.2 | 8.9 |
| CLP, 2X mannitol | 26.3 | 6.3 |

TABLE 2
Fluid Accumulation in CLP in Ileum

| Dose | Mean | Standard Deviation |
|---|---|---|
| CLP | 11.6 | 4.7 |
| CLP, 1X mannitol | 18.2 | 7.3 |
| CLP, 2X mannitol | 15.8 | 6.3 |

### Example 6

This example demonstrates that the addition of osmotic agents to cross-linked polyelectrolyte compositions (CLP) increases the absorption of fluid by the polyelectrolyte polymers in the gut.

In an exemplary method three rats per group were equilibrated on standard PMI 5012 rat chow and followed six days on altered diets and then compared to the last three days of each feeding period. The diets are 3% of the weight of the diet as the CLP prepared as described in Example 1 for one group and 3% CLP with 6% polyethylene glycol with a molecular weight of 3350 (PEG 3350) in the second group. Stool weights are shown in Table 3.

TABLE 3
Absorption of Fluid by CLP

| | CLP | PEG/CLP |
|---|---|---|
| Equilibrated Baseline Fecal Weight | 6.04 | 6.40 |
| Equilibrated Treatment Fecal Weight | 9.86 | 11.53 |
| Increase in Fecal Weight | 3.82 | 5.13 |
| P value treatment versus baseline | 4.6204E−06 | 3.80546E−06 |
| CLP dose (g) | 0.58 | 0.56 |
| Increased Feces per gram CLP | 6.63 | 9.18 |

Table 3 shows the results when the PEG 3350/CLP was fed. An increase in fecal output over the same CLP without the PEG 3350 that is statistically significant at the 0.036 p level was observed. These results demonstrate that different polyethylene glycol osmotic agents do increase the fluid removal by a polyelectrolyte.

### Example 7

This example demonstrates that an osmotic agent is capable of increasing fluid removal by a polyelectrolyte polymer from a mammal.

In an exemplary method, three 200 gram Sprague Dawley rats were placed in metabolic cages and placed on a diet of PMI 5012 with 4% of the weight of the diet composed of crosslinked polyacrylic acid prepared as described in Example 1. Another three 200 gram Sprague Dawley rats were placed in metabolic cages and fed a diet of PMI 5012 with 4% of the weight of the diet composed of crosslinked polyacrylic acid and 3% of the weight of the diet replaced by polyethylene glycol of molecular weight 950-1050. After equilibration on these diets, the rats consuming the polyacrylic acid passed 37% of their dietary intake as feces each day while the rats consuming the combination of polyethylene glycol and crosslinked polyacrylic acid averaged 61% of their dietary intake passed as feces each day. These results demonstrate that different polyethylene glycol osmotic agents do increase the fluid removal by a polyelectrolyte. Results are indicated in Table 4.

TABLE 4
CLP Administered with PEG 1000
Dose of CLP with PRG 1000

| | | | | | | | Na | Na | K | K |
|---|---|---|---|---|---|---|---|---|---|---|
| | Mean | SD | Mean | SD | Mean | SD | Removed | Removed | Removed | Removed |
| CLP Composition | Fluid | Fluid | Na | Na | K | K | (mean) | (SD) | (mean) | (SD) |
| 100% H-CLP as 4% of | 0.33 | 2.26 | 5.83 | 4.62 | 3.16 | 5.57 | | | | |
| diet | | | | | | | | | | |
| 100% H-CLP as 4% of | 1.94 | 2.70 | 5.85 | 3.45 | 5.07 | 7.23 | | | | |
| diet with 3% PEG 1000 | | | | | | | | | | |

### Example 8

This example demonstrates the removal of fluid (e.g., saline) and/or modulation (e.g., removal or donation) of ions (e.g., sodium and potassium) in rats administered a cross-linked polyelectrolyte polymer (CLP) with lubriprostone.

In an exemplary method, nine Sprague Dawley rats were placed after acclimatization, into individual metabolic cages and fed pulverized PMI 5012 rodent chow (1010 milligrams calcium, 1080 milligrams potassium, 210 milligrams magnesium, and 280 milligrams sodium per one-hundred grams chow). Rats were randomized into three groups to receive various forms of cross-linked polyelectrolyte polymer (“CLP”). The CLP was prepared as described in Example 1. Each rat serves as its own control, with a baseline period on the control diet and then a treatment period with CLP included in the diet. Rats were fed PMI 5012 rat chow for six days while being housed in metabolic cages. On the sixth day, each of five randomized groups of rats, has their diet replaced with CLP beads. Additionally, the rats were administered lubriprostone. Daily intake of food and water and daily output of feces and urine are recorded by weight. Fecal weight, fecal sodium and fecal potassium levels are calculated on days four, five, six, ten, eleven and twelve. Levels obtained on days four, five and six are compared to those obtained on days ten, eleven and twelve, respectively for each of the rat groups. For example, an amount of fluid removed is determined by subtracting the fecal weight on day ten by the fecal weight on day four. Similarly, the level of fecal sodium on day four is compared to the level of fecal sodium on day ten. Differences in fecal weight, fecal sodium and fecal potassium from the three comparisons from the three groups of rats are determined and mean and standard deviation are calculated. Results are presented in Table 5.

TABLE 5
CLP Administered with Lubriprostone
Dose of CLP with Lubriprostone

| | | | | | | | Na | Na | K | K |
|---|---|---|---|---|---|---|---|---|---|---|
| | Mean | SD | Mean | SD | Mean | SD | Removed | Removed | Removed | Removed |
| CLP Composition | Fluid | Fluid | Na | Na | K | K | (mean) | (SD) | (mean) | (SD) |
| 100% H-CLP as 5% of | 9.23 | 5.31 | 47.48 | 24.01 | 90.17 | 16.58 | | | | |
| diet | | | | | | | | | | |
| 100% H-CLP as 5% of | 11.21 | 2.69 | 45.18 | 11.74 | 83.03 | 11.12 | | | | |
| diet; 2.2 mcg lubriprostone | | | | | | | | | | |
| 100% H-CLP as 5% of | 13.20 | 4.09 | 54.48 | 12.70 | 74.64 | 28.13 | | | | |
| diet; 5.8 mcg lubriprostone | | | | | | | | | | |

### Example 9

This example demonstrates that some osmotic agents do not increase the removal of fluid from a mammal administered a superabsorbent polyelectrolyte polymer.

In an exemplary method, six normal human volunteers were placed into two groups and both received a controlled diet for six days followed by the identical diet with the addition of receiving oral doses of either 10 grams CLP or 10 grams CLP plus 10 grams mannitol per day. Stools were collected and weighed daily throughout the period. Table 6 details the results of the total six days of collection for each period.

TABLE 6
Stool Weight in the Presence or Absence of Mannitol

| | CLP | CLP/mannitol |
|---|---|---|
| Baseline | 1097.7 | 975.3 |
| Treatment | 2063.7 | 1564.7 |
| Increase in Fecal Weight | 966.0 | 589.3 |
| P | 0.033 | 0.170 |

Table 6 suggests that mannitol given concomitantly with CLP did not significantly increase fecal output (p 0.42 for difference between 966 and 589) and appears to have decreased fecal output. These results suggest that mannitol does not increase fluid removal and may decrease fluid removal upon passage through the entire gastrointestinal tract.

### Example 10

A clinical trial of an exemplary CLP was conducted as described in Example 9. Objectives of the clinical trial included: to determine if the addition of mannitol, an osmotic agent, enhances stool weight increase compared with baseline period and with CLP administered alone (see Example 9 for results); to determine the amount of fluid absorbed per gram of CLP administered as assessed by stool weight compared with baseline period in a fasted state; and to determine the effect of the capsule enteric-coating on removal of calcium, magnesium, and potassium.

Primary endpoints included: net sodium balance compared among treated and control groups. Secondary endpoints included: change in stool weight compared among treated and control groups (see Example 9 for results); net balance of calcium, magnesium, potassium, iron, copper, zinc and phosphorous compared among treated and control groups; fluid consumed and excreted in the treated groups compared with the control group; and safety and tolerability based upon review of vital signs, clinical safety labs and adverse events.

An open-label, non-randomized, multiple-dose study in six healthy subjects divided into two groups of three subjects in each group was conducted. Subjects participate in a six day baseline period during which diet was controlled and stool weight and fluid balance was determined. The treatment period began on day seven. During the treatment period, dosing with CLP capsules took place, diet was controlled and stool weight and fluid balance was determined. The subjects selected had the ability to swallow up to 27 capsules each day of the study that included CLP dosing.

Subjects were placed into one of two groups on day six based upon the previous five days of stool weights, so that the average daily stool weight was approximately equal across groups. Group 1 received CLP in enteric-coated capsules. Group 2 received CLP in enteric-coated capsules mixed with mannitol. CLP with and without mannitol was supplied in hard size 00 HPMC enteric-coated capsules and sent as a finished dosage.

The encapsulated CLP used in the study incorporates an enteric coating that is reported to dissolve at approximately pH 5.5 while being insoluble at more acidic pH values. Gastric pH is usually below pH 2 while the stomach is empty, rises to about pH 7 with the ingestion of food, and falls back to about pH 2 within ten to fifteen minutes of the start of a meal as gastrointestinal hormones are secreted, causing the secretion of large amounts of hydrochloric acid. In the duodenum a higher pH is encountered and the coating dissolves. Since passage through the duodenum takes about five minutes, the CLP is expected to be exposed to the intestinal fluid first in the upper jejunum and absorb 90% of its fluid capacity during the ninety minutes of small bowel transit.

During the Treatment Period, oral total daily doses of ten grams CLP was divided into four doses and administered for six days, for a total of twenty-four consecutive doses in the treatment period. For Group I, the CLP dose was milled with 0.73 grams per capsule and divided into four doses in capsules. For Group 2, the 10 gram CLP was milled with 0.365 grams per capsule and with 10 grams mannitol in the same capsule (0.365 grams per capsule) and divided in four doses in capsules.

A standardized diet was administered throughout the study, including a six-day baseline period during which no CLP dosing occurs. All meals provided to the subjects are controlled for the number of calories, fat and fiber content. CLP was administered in a fasted state, at least one hour prior to each of four meals/snacks (e.g., breakfast, lunch, dinner and snack) and administered with water. Subjects were restricted from additional fluids for one hour pre and post dose.

All urine was collected separately for each twenty-four hour period, measured, and then discarded except for study day five and study day eleven when the urine for the entire day was pooled to allow an aliquot to be sent for potassium, magnesium and calcium analysis.

All feces were collected and each sample was individually weighed and the color and consistency of the stool noted. The weights were added together to determine the total fecal weight resulting from each day's intake. The entire weight of stool between the color markers was also totaled separately for both the baseline period and the experimental period.

All feces eliminated after consumption of the first controlled meal were collected as individual samples in tared collection containers, labeled, accurately weighed, then frozen and stored at or below −20° C. Individual stools during each twenty-four hour interval were collected and stored separately. Protocol number, subject number, study day, date and time of sample collection, and weight of sample and container was clearly and permanently indicated on collection containers for each individual stool. Sample time, presence of beads, color, consistency and weight were also recorded in the medical record. Fecal and urine collections from days five and eleven were submitted for analysis of calcium, magnesium, and potassium.

No formal statistical analysis of fecal weights was performed. Fecal weight data was summarized using descriptive statistics (mean, standard deviation, median, minimum and maximum) as appropriate. The cumulative weight of all stool samples excreted during the Baseline Period was compared to the cumulative weight of the stool samples excreted during the Treatment Period, by calculating change from baseline values for each subject. Comparisons are made both within and among the two treatment groups.

Summary statistics for fecal and urine concentrations of calcium, magnesium, and potassium on day five and day eleven (as well as for changes from Day 5 to Day 11) were presented by treatment group. Fluid balance data was summarized using descriptive statistics (e.g., mean, standard deviation, median, minimum and maximum) as appropriate. Fluid intake and output were measured for each twenty-four hour period during the study. These measurements were compared between baseline and treatment periods.

Change in fecal metal excretion (e.g., sodium, potassium, magnesium and calcium) for varying administrations of CLP are shown in Table 7.

TABLE 7
Change in Fecal Metal Excretion (mg/day)

| CLP | Na | K | Mg | Ca |
|---|---|---|---|---|
| Milled, uncoated | 1279 | 82 | 1768 | 1245 |
| Milled, uncoated; mannitol | 803 | −8 | 448 | 369 |
| Beads, uncoated; mannitol | 832 | 64 | 715 | 843 |
| Milled, coated pH 5.5 | 1445 | 119 | 1133 | 1518 |
| Milled, coated pH 5.5; mannitol | 1469 | 45 | 579 | 1335 |

### Example 11

A clinical trial of an exemplary CLP was conducted. Objectives of the clinical trial included: to determine if the addition of mannitol, an osmotic agent, enhances stool weight increase compared with baseline period and with CLP administered alone; to determine the amount of fluid absorbed per gram of CLP administered as assessed by stool weight compared with baseline period in both a fed and fasted state; and to determine the impact of CLP on the potential removal of trace elements such as Ca, Mg and K.

Primary endpoints included: net sodium balance compared among treated and control groups. Secondary endpoints included: change in stool weight compared among treated and control groups; net balance of calcium, magnesium, potassium, iron, copper, zinc and phosphorous compared among treated and control groups; fluid consumed and excreted in the treated groups compared with the control group; and safety and tolerability based upon review of vital signs, clinical safety labs and adverse events.

For this clinical trial, an open-label, non-randomized, multiple-dose study in eighteen healthy subjects divided into six groups of three subjects in each group was conducted. Subjects were placed into one of six groups on day six, based upon the previous five days of stool weights, so that the average daily stool weight is approximately equal across groups. A summary of treatment groups is indicated in Table 8.

TABLE 8
Treatment Groups

| Group | CLP Details | Mannitol | Fasted/Fed |
|---|---|---|---|
| 1 | milled, 212μ-500μ | No | Fasted |
| | particles | | |
| 2 | milled, 212μ-500μ | Yes, with CLP | Fasted |
| | particles | | |
| 3 | 500μ-710μ beads | Yes, separate | Fasted |
| | | uncoated capsules | |
| 4 | 710μ-1000μ beads | Yes, separate | Fasted |
| | | uncoated capsules | |
| 5 | milled, 212μ-500μ | No | Fed |
| | particles | | |
| 6 | milled, 212μ-500μ | Yes, with CLP | Fed |
| | particles | | |

CLP (milled) with and without mannitol (Groups 1, 2, 5 and 6) was in size 00 HPMC capsules that are uncoated. CLP (beads) and mannitol alone were supplied in bottles so that they may put into size 00 HPMC capsules (Groups 3 and 4).

CLP was administered according to treatment assignment (Groups 1, 2, 3, and 4) in a fasted state, at least one hour prior to each of four meals/snack and administered with water. Subjects were restricted from additional fluids for one hour pre and post dose. For Groups 5 and 6, CLP is administered in a fed state, thirty minutes following the end of a meal and evening snack, administered with water and without any fluid restriction.

All meals provided to the subjects were controlled for the number of calories, fat and fiber content; and the same meals is provided on corresponding days of Baseline and Treatment periods.

All urine was collected separately, weighed, and then discarded except for Study Day 5 and Study Day 11 when the urine for the entire day was pooled to allow an aliquot to be sent for K, Mg, and Ca analysis. All feces were collected in separate containers.

Each sample was individually weighed and the color and consistency of the stool noted. The weights were added together to determine the total fecal weight resulting from each day's intake. The entire weight of stool between the color markers was also totaled separately for both the baseline period and the experimental period. Stool was also examined for the presence of beads during the treatment period. Results from the measurement of stool weight in groups receiving mannitol similar in kind to those in Example 9 again suggest that mannitol does not increase fluid removal and may decrease fluid removal upon passage through the entire gastrointestinal tract.

During the Treatment Period, oral total daily doses of ten grams CLP was divided into four doses and administered with water for six days, for a total of twenty-four consecutive doses in the treatment period. Doses were given within ten minutes of the scheduled time for each subject.

All feces eliminated after consumption of the first controlled meal were collected as individual samples in tared collection containers, labeled, accurately weighed, then frozen and stored at or below −20° C. Individual stools during each twenty four hour interval are collected and stored separately. Fecal collections from Days 5 and 11 are submitted for analysis of calcium, magnesium, and potassium.

Every urine specimen was collected and weighed. For Study Days 5 and 11 the urine is pooled for that day and analyzed for K, Mg, and Ca.

Change in fecal metal excretion (e.g., sodium, potassium, magnesium and calcium) for varying administrations of CLP are shown in Table 9.

TABLE 9
Change in Fecal Metal Excretion (mg/day)

| CLP Details | Na | K | Mg | Ca |
|---|---|---|---|---|
| Milled, uncoated | 5.56 | 0.21 | 7.27 | 3.11 |
| Milled, uncoated; mannitol | 3.49 | −0.02 | 1.84 | 0.92 |
| Beads, uncoated; mannitol | 3.62 | 0.16 | 2.94 | 2.10 |
| Milled, coated pH 5. 5 | 6.28 | 0.31 | 4.66 | 3.79 |
| Milled, coated pH 5.5; mannitol | 6.39 | 0.12 | 2.38 | 3.33 |

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method of removing fluid from a subject, comprising:
a) administering a cross-linked polyelectrolyte polymer that absorbs about 20-fold or more of its mass in saline; and
b) administering an agent that increases the fluid in the intestine of the subject.

2. The method of claim 1 further comprising identifying a subject in need of fluid removal.

3. The method of claim 1, wherein the polyelectrolyte polymer is polyacrylate.

4. The method of claim 1, wherein the agent increases the fluid in the small intestine or colon or both the small intestine and colon.

5. The method of claim 1, wherein the agent is selected from the group consisting of: a osmotic agent, an inhibitor of intestinal sodium transport and an agent which increases sodium secretion into the intestine.

6. The method of claim 1, wherein the agent is a non-fermentable osmotic agent.

7. The method of claim 1, wherein the agent is selected from the group consisting of: mannitol, polyethylene glycol and lubiprostone.

8. The method of claim 7, wherein polyethylene glycol has a molecular weight between 400 and 10,000 Daltons.

9. The method of claim 7, wherein the polyethylene glycol has a molecular weight between 400 and 4000 Daltons.

10. The method of claim 1, wherein the agent is a non-absorbed sugar.

11. The method of claim 1, wherein the agent is a poorly absorbed salt.

12. The method of claim 1, wherein the agent is an irritant.

13. The method of claim 1, wherein the agent inhibits sodium transport in the intestinal wall.

14. The method of claim 1, wherein the agent stimulates fluid secretion into the intestine.

15. The method of claim 1, wherein the cross-linked polyelectrolyte polymer that absorbs more than 60-fold, 70-fold, 80-fold, 90-fold, 100-fold, 110-fold or more of its mass in aqueous saline.

16. The method of claim 1, wherein the cross-linked polyelectrolyte polymer is substantially free of soluble polyacrylic acid polymer.

17. The method of claim 1, wherein the cross-linked polyelectrolyte polymer comprises one or more bound counterions.

18. The method of claim 1, wherein the cross-linked polyelectrolyte polymer comprises one or more inorganic counterions.

19. The method of claim 18, wherein the inorganic counterion is hydrogen.

20. The method of claim 1, wherein the cross-linked polyelectrolyte polymer comprises one or more bound organic counterions.

21. The method of claim 1, wherein the cross-linked polyelectrolyte polymer comprises one or more inorganic counterions and at least one or more organic counterions.

22. The method of claim 1, wherein the cross-linked polyelectrolyte polymer is encapsulated in a capsule.

23. The method of claim 22, wherein the capsule is coated with an enteric or delayed release coating.

24. The method of claim 1, wherein the cross-linked polyelectrolyte polymer is substantially in the shape of a disrupted sphere or ellipsoid.

25. The method of claim 1, wherein the cross-linked polyelectrolyte polymer is disrupted by milling or crushing.

26. The method of claim 1, wherein the cross-linked polyelectrolyte polymer is directly administered to the small intestine.

27. The method of claim 26, wherein the cross-linked polyelectrolyte polymer is directly administered to the jejunum.

28. The method of claim 1, wherein the cross-linked polyelectrolyte polymer is directly administered to the colon.

29. The method of claim 1, wherein the cross-linked polyelectrolyte polymer is administered orally.

30. The method of claim 1, wherein the subject has cardiac disease.

31. The method of claim 30, wherein the cardiac disease is congestive heart failure.

32. The method of claim 1, wherein the subject has kidney disease.

33. The method of claim 32, wherein the kidney disease is nephrosis, nephritis or end stage renal disease (ESRD).
